(19) United States
(12) Patent Application Publication
(10) Pub. No.: US 2014/0083433 A1
(43) Pub. Date: Mar. 27, 2014

(54) INFLATABLE MULTILEVEL VAGINAL PESSARY DEVICE

(71) Applicant: Suzanne Lee Lowry, Atlanta, GA (US)

(72) Inventor: Suzanne Lee Lowry, Atlanta, GA (US)

(21) Appl. No.: 13/625,831

(22) Filed: Sep. 24, 2012

(51) Int. Cl. A61F 6/08

(52) U.S. Cl. 128/836; 128/834

(57) ABSTRACT

A multilevel, inflatable vaginal pessary device is soft, malleable and has a gentle gripping surface to assure retention and proper placement. The pessary device can offer options for pressure, either apically toward the cervix or anteriorly toward the bladder or posteriorly toward the rectum, via an inner silicone elastomeric (such as Silastic® by Dow Corning Corporation) balloon that can variably be filled with air from a syringe attached to a luer lock tip in the balloon. Exposure of the balloon is achieved by the physical opening potential of the fenestrated rings by cutting along guide lines in the pessary sleeve. The physician has multiple options for customizing the device to remedy the patient's prolapse.

### BACKGROUND OF THE INVENTION

The present invention relates to vaginal pessary devices and, more particularly, to an inflatable, multilevel vaginal pessary device.

Pelvic organ prolapse occurs when the tissues and muscles that hold pelvic organs in place are stretched and/or weakened. The organs move from their natural position to press against the vaginal wall. The condition is strongly linked to labor and childbirth, but it may also be related to other conditions that cause increased pressure in the abdomen, such as obesity, prior abdominal and pelvic surgeries, respiratory problems with chronic coughing and constipation. In some women, pelvic prolapse can be painful or uncomfortable and can be associated with embarrassing hygiene problems, such as leaking urine or difficulty with bowel function.

A vaginal pessary is a removable device that is placed in the vagina and is designed to support areas of the prolapsed pelvic organs. There exists a variety of available pessaries. Little modification or improvement of these devices has occurred over the past 40 or more years. Some pessary devices are made of rubber or plastic or silicone-based materials. Some are inflatable, with variable shapes; doughnut or square or round with knobs and different shaped frames of plastic. Pessaries should be fitted by a health care professional and experimentation may be needed to find the right size and fit. The patient is taught to remove, clean and reinsert the pessary on a schedule which is dependent on the type of device or the type of pelvic prolapse.

Pessaries are used as a non-surgical approach to the treatment of pelvic prolapse. Frequently, they are used temporarily in young women during pregnancy and removed once the uterus has enlarged and remains fixed in place. They are often used as the non-surgical option of choice for high risk patients who may not tolerate a surgical procedure or for non-ambulatory elderly patients for whom surgery is not an option and hygiene may be improved after placement of the device.

Complications of a “fixed” device include vaginal sores, bleeding and, in severe cases, fistula formulation. Complications may be minimized in a properly fitted pessary or with a device that allows pressure adjustment. In menopausal women, estrogen cream or a lubricant is often used to place and remove the pessary. The cost of ambulatory care for pelvic prolapse has shown a steady and significant increase. In 2005-2006, Over $412 million was spent addressing this problem. With increasing prevalence in the growing population of elderly women, the problem is likely to expand.

Currently available pessaries are fixed with minimal flexibility and have no gripping surface. Available pessaries offer little or no support to the entire vaginal surface or require such a large device that they cause erosion and bleeding while rarely solving the problem of prolapse. Because they are a fixed shape and many are made from a non-malleable material, they are painful to place and difficult to remove. They can fall out or cause damage to the vaginal tissue.

As can be seen, there is a need for a precise device that offers multilevel adjustable support, yet is soft and malleable and has a gentle gripping surface to assure retention and accurate placement.

### SUMMARY OF THE INVENTION

In one aspect of the present invention, a pessary device comprises a sleeve having open ends; a lip disposed on each end of the sleeve; an adjustable aperture ring disposed on each end of the sleeve, the adjustable aperture ring capable of adjusting the diameter of the open ends; and a sleeve body aperture formed along a side of the sleeve.

In another aspect of the present invention, a pessary device comprises a sleeve having open ends; a lip disposed on each end of the sleeve; an adjustable aperture ring disposed on each end of the sleeve, the adjustable aperture ring capable of adjusting the diameter of the open ends, the adjustable aperture ring including one or more score lines concentrically disposed thereupon to adjust an inside diameter of the adjustable aperture ring; a sleeve body aperture formed along a side of the sleeve; a plurality of grip protrusions disposed along the sleeve; and an aperture opening disposed along one side of the sleeve.

These and other features, aspects and advantages of the present invention will become better understood with reference to the following drawings, description and claims.

### BRIEF DESCRIPTION OF THE DRAWINGS

FIG. 1 is a perspective view of a pessary device according to an exemplary embodiment of the present invention;

FIG. 2 is a bottom perspective view of the pessary device of FIG. 1;

FIG. 3 is a side perspective view of the pessary device of FIG. 1;

FIG. 4 is a cross-sectional view taken along line 4-4 of FIG. 1; and

FIG. 5 is a cross-sectional anatomical view showing the pessary device of FIG. 1 placed in the vagina of a patient.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a multilevel, inflatable vaginal pessary device that is soft, malleable and has a gentle gripping surface to assure retention and proper placement. The pessary device can offer options for pressure, either apically toward the cervix or anteriorly toward the bladder or posteriorly toward the rectum, via an inner silicone elastomeric (such as Silastic® by Dow Corning Corporation) balloon that can variably be filled with air from a syringe attached to a luer lock tip in the balloon. Exposure of the balloon is achieved by the physical opening potential fenestrated rings by cutting along guide lines in the pessary sleeve. The physician has multiple options for customizing the device to remedy the patient's prolapse.

The pessary device of the present invention offers superior pelvic floor support and prolapse resolution either postponing or eliminating the need for major surgery. The combination of a multilevel two ringed support system with grips offers greater global support for the vaginal vault than any currently available device. Add to that the ability to tailor the device by opening individual apertures and then by placing the balloon in the sleeve for pressure to direct, specific areas for support to resolve each patient's prolapse issue. The pessary device of the present invention, in a compromised patient (confined), solves hygiene issues and restores a patient's dignity. Different sizes of the pessary device allow the physician to further tailor the whole device system by adjusting the amount of air that fills the balloon and by adjusting the aperture size openings. This allows each device to be specifically adjusted to each patient's need with general and specific vaginal support, apically, anteriorly, posteriorly or in multiple areas if called for in any particular patient.

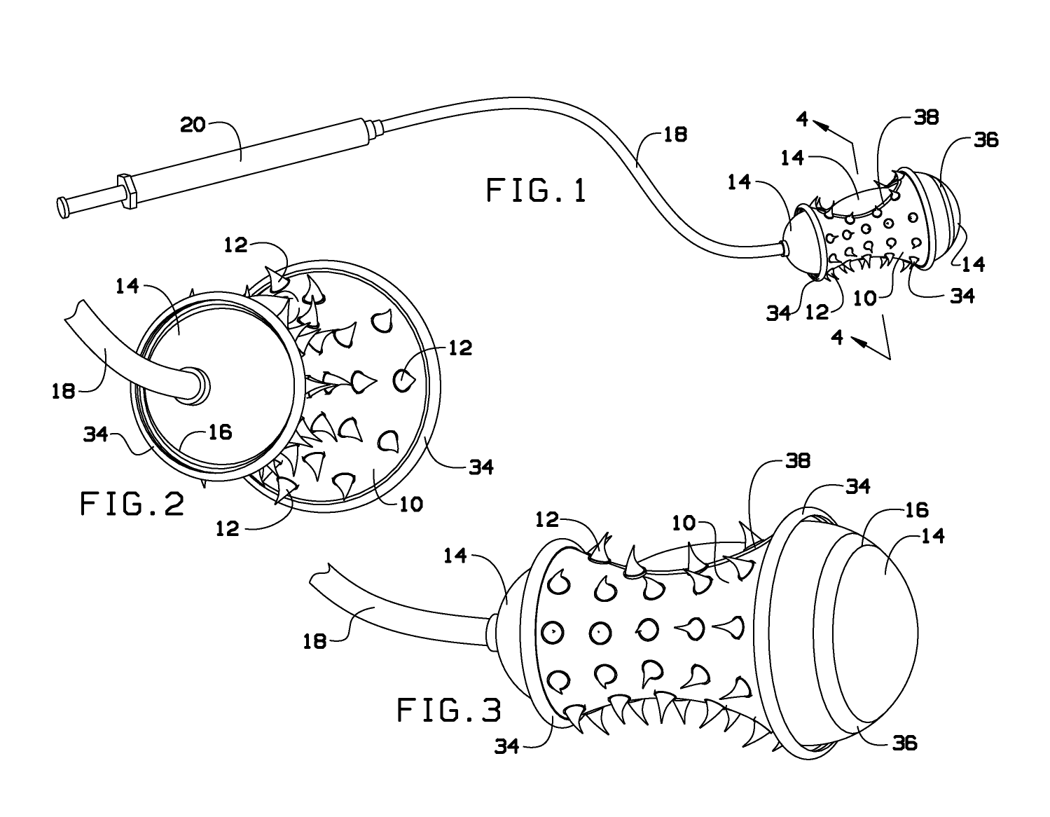

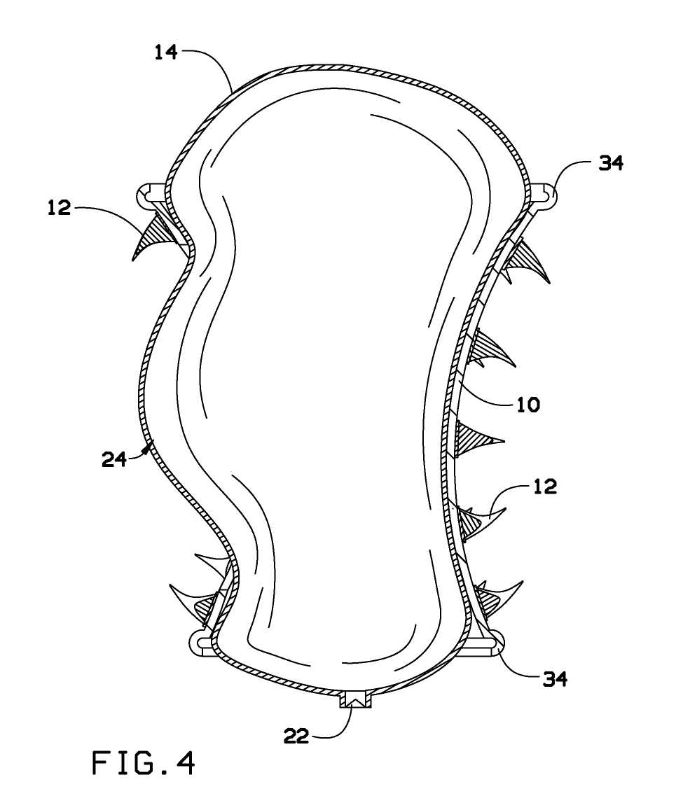

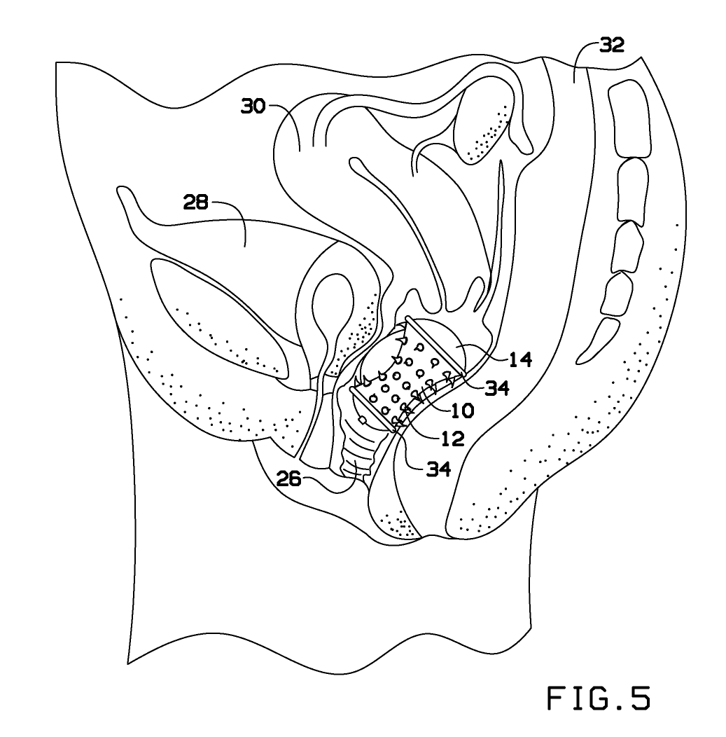

Referring now to FIGS. 1 through 5, a pessary device can include a sleeve 10, typically a soft rubber sleeve fitted with a latex-free silicone balloon 14. A plurality of grip protrusions 12 can be disposed along the sleeve 10. The grip protrusions 12 can be from about 0.3 to about 0.8 cm in height, typically about 0.5 cm in height, and about 0.2 to about 0.3 cm thick. The sleeve 10 can be from about 3 cm to about 7 cm in length, typically about 5 cm in length. The sleeve 10 can be from formed from a material about 0.5 cm thick, for example.

A sleeve 10 can be a tubular member with openings on each end thereof. Aperture rings 36 can be connected to a lip 34 of each opening. The aperture rings 36 can be interconnected, end-to-end, such that the opening becomes progressively smaller (typically by about 1 cm each) with each aperture ring 36 connected. A physician can choose a size of the opening by separating the appropriate aperture rings 36 and cutting along a score line 16 therebetween. As the balloon 14 is inflated with air 24, it can extend from the lips 34 on each end of the sleeve 10 consistent with the physician determined opening size.

The balloon 14 can be filled with air using a standard syringe 20 and luer-lock tubing 18. Typically the balloon 14 can hold from about 125 to about 175 cc or air, usually about 150 cc of air. A lock valve 22 can be disposed to allow the user to attach and remove the luer-lock tubing 18 from the balloon 14 in the pessary device. The user can use the tubing for inflation or deflation of the balloon 14.

The sleeve 10 can include an aperture opening 38 along one side thereof. This opening 38 can allow the balloon 14 to protrude therefrom, as shown in FIG. 1. This opening 38 can be turned and inflated to resolve prolapse in an anterior wall (cystocele) or posterior wall (rectocele).

In some embodiments, the pessary device can be used without the balloon 14 for gentle support.

The pessary device of the present invention can be made of various suitable materials, including a medical grade silicone, rubber or soft plastic sleeve encasing a proximal and distal ring (of various sizes, such as small (5 cm diameter), medium (7 cm diameter) and large (9 cm in diameter)) with a surface that contains soft gripping exvaginations. Aperture rings can be formed on the ends of the sleeve that can be opened by cutting away part of the sleeve along the imprinted guide lines with a scissor to expose the balloon for specific customization toward the area of prolapse. The components can be manufactured with standard manufacturing techniques, such as injection molding.

The pessary device can be placed in the vagina 26 and can be configured to provide support for the uterus 30, bladder 28, or rectum 32, for example.

The pessary device of the present invention allows for superior, specific mechanical support of each area of pelvic floor prolapse resolution. The variety of aperture size openings and the soft rubber sleeve assures better patient safety while offering specific support. The device of the present invention gives the patient and the health care provider the option to use a single device to specifically address the area of prolapse and may offer improved control of adjacent organs while offering comfort and support.

It should be understood, of course, that the foregoing relates to exemplary embodiments of the invention and that modifications may be made without departing from the spirit and scope of the invention as set forth in the following claims.

What is claimed is:

1. A pessary device comprising:
a sleeve having open ends;
a lip disposed on each end of the sleeve;
an adjustable aperture ring disposed on each end of the sleeve, the adjustable aperture ring capable of adjusting the diameter of the open ends; and
a sleeve body aperture formed along a side of the sleeve.

2. The pessary device of claim 1, wherein the adjustable aperture ring includes one or more score lines concentrically disposed thereupon to adjust an inside diameter of the adjustable aperture ring.

3. The pessary device of claim 1, further comprising a plurality of grip protrusions disposed along the sleeve.

4. The pessary device of claim 1, further comprising an aperture opening disposed along one side of the sleeve.

5. The pessary device of claim 1, further comprising a balloon disposed in the sleeve.

6. The pessary device of claim 5, further comprising an inflation mechanism allowing the control of air into and out of the balloon.

7. The pessary device of claim 6, wherein the inflation mechanism includes a luer-lock tubing having a syringe on one end thereof.

8. A pessary device comprising:
a sleeve having open ends;
a lip disposed on each end of the sleeve;
an adjustable aperture ring disposed on each end of the sleeve, the adjustable aperture ring capable of adjusting the diameter of the open ends, the adjustable aperture ring including one or more score lines concentrically disposed thereupon to adjust an inside diameter of the adjustable aperture ring;
a sleeve body aperture formed along a side of the sleeve;
a plurality of grip protrusions disposed along the sleeve; and
an aperture opening disposed along one side of the sleeve.

9. The pessary device of claim 8, further comprising a balloon disposed in the sleeve.

10. The pessary device of claim 9, further comprising an inflation mechanism allowing the control of air into and out of the balloon.

11. The pessary device of claim 10, wherein the inflation mechanism includes a luer-lock tubing having a syringe on one end thereof.
